(19) United States
(12) Patent Application Publication
(10) Pub. No.: US 2026/0026957 A1
(43) Pub. Date: Jan. 29, 2026

(54) OSTOMY APPLIANCE WITH RADIAL ELECTRODE PARTS

(71) Applicant: Coloplast A/S, Humlebaek (DK)

(72) Inventors: Lars Molzen, Alleroed (DK); Anders Grove Sund, Alleroed (DK)

(21) Appl. No.: 18/994,085

(22) PCT Filed: Jul. 13, 2023
(86) PCT No.: PCT/DK2023/050188

§ 371 Date: Jan. 14, 2025

(30) Foreign Application Priority Data
Jul. 14, 2022 (DK) PA202270380

(51) Int. Cl. A61F 5/44; A61F 5/443

(52) U.S. Cl. CPC A61F 5/4404; A61F 5/443

(57) ABSTRACT

An ostomy appliance for attachment to the skin surface of a user, comprising a support layer, an adhesive layer, and a plurality of primary electrode paths for forming one or more sensors, a first pair of the plurality of primary electrode paths comprises a first electrode path and a second electrode path each including a respective radial part extending towards the stomal opening in a first angular section, and a second pair of the plurality of primary electrode paths comprises at least the third electrode path, each electrode path of the second pair includes a respective radial part extending towards the stomal opening in a second angular section.

### TECHNICAL FIELD

The present disclosure relates to an ostomy appliance, a method of manufacturing an ostomy appliance, and a method of adapting a stomal opening of an ostomy appliance.

### BACKGROUND

Stomal output often contains body fluids and visceral contents that are aggressive to both the skin of a user and to ostomy devices. These have a detrimental effect on the efficiency and integrity of the adhesive materials that are applied to attach the ostomy device to the user's skin surface. For users in general, safe, reliable and efficient ostomy devices are evidently highly desirable.

However, a particularly major and persistent concern of a large population of ostomists continues to be failure of the base plate adhesive attaching the ostomy appliance to the user's skin surface, because such failure almost inevitably leads to embarrassing and stigmatising leakage incidents. Such incidents in turn are known from several user interviews to lead to a reduced quality-of-life feeling. Adhesive failure of the base plate adhesive can result from various reasons. Most often, a leakage incident is caused by stomal output entering between the proximal surface of the base plate and the user's skin, e.g. due to less-than-optimal attachment of the base plate to the skin arising from e.g. uneven skin surface, skin folds, or a complex stoma geometry. This undesirable progression of stomal output “underneath” the adhesive leads to deterioration and/or weakening of the adhesive material carrying the weight and providing the seal of the ostomy appliance. Often, such failure happens surprisingly fast and is only detectable for the user once the failure has already become so severe that leakage occurs, requiring immediate change of the ostomy appliance and possibly also of the user's clothes.

In other instances, the primary factor of adhesive failure is simply a question of how much time has elapsed since the base plate of the ostomy appliance was first applied to the user's skin surface. In addition to the output from the stoma itself, the peristomal skin surface continuously secretes some moisture (e.g. sweat). To mitigate this, most often adhesives of base plates for ostomy devices include hydrocolloid materials which are capable of absorbing high levels of moisture, thereby stabilizing the polymer matrix of the adhesive material and prolonging the lifetime (“wear time”) of the base plate. However, eventually the adhesion capability of the base plate no longer can support the force exerted on the base plate from the load of the output collecting bag, and the appliance must be replaced.

As there can be considerable differences in the severity and/or speed by which adhesive failure and potentially leakage occur, which differences at least to some extent are correlated to various factors including those presented above, a mere indication that failure or leakage is imminent, or that it has already occurred, fails to represent a reliable and satisfactory solution to the problem of avoiding sudden embarrassing and stigmatising leakage incidents in ostomy appliances. In other words, the users of ostomy appliances could greatly benefit from an appliance solution which provides them with better guidance and options regarding how and—not least—how quickly to react to beginning failure or leakage of the adhesive of the base plate of the appliance. More generally, ostomists and health care professionals alike would welcome improvements in ostomy devices to reduce or eliminate the occurrence of sudden leakage incidents.

### SUMMARY

On this background, it may be seen as an object of the present disclosure to provide an ostomy appliance comprising for example a sensor patch and/or a base plate for facilitating reliable and/or improved detection of risk of failure of an ostomy appliance and/or improved detection of risk of leakage. Another object of the present disclosure is to provide a method of manufacturing such an ostomy appliance. A further object of the present disclosure is to provide a method of adapting an ostomy appliance to the stoma of a user.

One or more of these objects may be met by aspects of the present disclosure as described in the following.

A first aspect of this disclosure relates to an ostomy appliance for attachment to the skin surface of a user, wherein the ostomy appliance is adapted to form a stomal opening with a centre point, the stomal opening being configured to allow passage of output through the stomal opening and into an ostomy pouch attached to the ostomy appliance, the ostomy appliance comprising:
a support layer with a distal surface and a proximal surface;
an adhesive layer including a distal surface and a proximal surface, the distal surface being arranged on the proximal surface of the support layer; and
a plurality of primary electrode paths for forming one or more sensors, the plurality of primary electrode paths comprising at least a first electrode path, a second electrode path, and a third electrode path, wherein the plurality of primary electrode paths is arranged on the proximal surface of the support layer and covered by, and preferably in contact with, the adhesive layer;
   wherein a first pair of the plurality of primary electrode paths comprises the first electrode path and the second electrode path, wherein each electrode path of the first pair includes a respective radial part extending in a substantially radial direction towards the stomal opening in a first angular section of a plurality of angular sections of the adhesive layer, the radial parts of the first pair being configured for allowing the provision of a first signal indicative of liquid on the proximal surface of a first angular section of the plurality of angular sections of the adhesive layer and/or indicative of a moisture level in the first angular section of the plurality of angular sections of the adhesive layer, wherein a second pair of the plurality of primary electrode paths comprises at least the third electrode path, each electrode path of the second pair includes a respective radial part extending in a substantially radial direction towards the stomal opening in the second angular section of the plurality of angular sections of the adhesive layer, the radial parts of the second pair being configured for allowing the provision of a second signal indicative of liquid on the proximal surface of a second angular section of the plurality of angular sections of the adhesive layer and/or indicative of a moisture level in the second angular section of the plurality of angular sections of the adhesive layer.

Providing pairs of electrode paths being more indicative of liquid or moisture in a given angular section allows the ostomy appliance to advantageously indicate to the user that the risk of a leak in a particular angular section is increased relative to other angular sections. Such information may allow the user to decide whether replacement of the ostomy appliance is necessary. Further, such information may allow the user to improve the attachment of the ostomy appliance to the skin surface of the user. For example, if a moisture or liquid is detected in a consistent angular section, the user may take additional care to attach the ostomy appliance.

Additionally or alternatively, the plurality of primary electrode paths may be configured for providing the two or more signals by measuring the electrical resistance between one electrode path of the plurality of primary electrode paths and another electrode path of the plurality of primary electrode paths.

Additionally or alternatively, the two or more signals may preferably be indicative of a liquid (e.g., a leak) or moisture (e.g., an elevated moisture level) between the proximal surface of the adhesive layer and the support layer in a respective angular section of the plurality of angular sections.

In the context of the present disclosure, the term “substantially radial direction” refers to a direction which is generally angled towards a centre point of the stomal opening.

Additionally or alternatively, the first signal may be more indicative of, preferably more correlated with, liquid on the proximal surface of a first angular section of the plurality of angular sections of the adhesive layer and/or more indicative of a moisture level in the first angular section of the plurality of angular sections of the adhesive layer than of the second angular section of the adhesive layer, preferably than of any other of the angular sections of the adhesive layer, and/or wherein the second signal may be more indicative of, preferably more correlated with, liquid on the proximal surface of the second angular section of the plurality of angular sections of the adhesive layer and/or more indicative of a moisture level in the second angular section of the plurality of angular sections of the adhesive layer than of the first angular section of the adhesive layer, preferably than of any other of the angular sections of the adhesive layer.

Additionally or alternatively, the radial parts of the first electrode path and the second electrode path may extend in the first angular section. The radial part(s) of at least one of the first electrode path and the second electrode path may not extend in the second angular section. Preferably, the radial part(s) of at least one of the first electrode path and the second electrode path may not extend in any other of the angular sections than the first angular section.

Additionally or alternatively, the radial parts of both of the electrode paths of the second pair of the plurality of primary electrode paths may extend in the second angular section. The radial part(s) of at least one electrode path of the second pair of the plurality of primary electrode paths may not extend in the first angular section. Preferably the radial part(s) of at least one electrode path of the second pair of the plurality of primary electrode paths may not extend in any other of the angular sections than the second angular section.

Accordingly, moisture or liquid in other angular sections than the respective angular section of the electrode path pair has reduced effect on the signal. For example, moisture or liquid in the second angular section does not significantly influence the signal provided by the first pair of electrode paths since only at most one of the first and second electrode pairs extend in the second angular section.

Additionally or alternatively, the second pair of the plurality of primary electrode paths may comprise the first electrode path and the third electrode path, and a third pair of the plurality of primary electrode paths may comprise the second electrode path and the third electrode path. Each electrode path of the third pair may include a respective radial part extending in a substantially radial direction towards the stomal opening in a third angular section of the plurality of angular sections of the adhesive layer. The radial parts of the third pair may be configured for allowing the provision of liquid on the proximal surface of the third angular section of the plurality of angular sections of the adhesive layer and/or indicative of a moisture level in the third angular section of the plurality of angular sections of the adhesive layer. Preferably, the third signal may be more indicative of, preferably more correlated with, liquid on the proximal surface of the third angular section of the plurality of angular sections of the adhesive layer and/or more indicative of a moisture level in the third angular section of the plurality of angular sections of the adhesive layer than of the first angular section of the adhesive layer and the second angular section of the adhesive layer, preferably than of any other of the angular sections of the adhesive layer.

Additionally, both electrode paths of the third pair of the plurality of primary electrode paths may extend in the third angular section. At least one electrode path of the third pair of the plurality of primary electrode paths may not extend in any one of the first angular section and the second angular section. Preferably at least one of the third pair of the plurality of primary electrode paths does not extend in any other of the angular sections than the third angular section.

Additionally or alternatively, the plurality of primary electrode paths includes a fourth electrode path. A fourth pair of the plurality of primary electrode paths may comprise the first electrode path and the fourth electrode path. Each electrode path of the fourth pair may include a respective radial part extending in a substantially radial direction towards the stomal opening in a fourth angular section of the plurality of angular sections of the adhesive layer. The radial parts of the fourth pair may be configured for allowing the provision of a fourth signal indicative of liquid on the proximal surface of the fourth angular section of the adhesive layer. The fourth signal may preferably be more indicative of liquid on the proximal surface of the fourth angular section of the adhesive layer than of liquid on the proximal surface of any other of the plurality of angular sections of the adhesive layer. A fifth pair of the plurality of primary electrode paths may comprise the second electrode path and the fourth electrode path. Each electrode path of the fifth pair may include a respective radial part extending in a substantially radial direction towards the stomal opening in a fifth angular section of the plurality of angular sections of the adhesive layer. The radial parts of the fifth pair may be configured for allowing the provision of a fifth signal indicative of liquid on the proximal surface of the fifth angular section of the adhesive layer. The fifth signal may preferably be more indicative of liquid on the proximal surface of the fifth angular section of the adhesive layer than of liquid on the proximal surface of any other of the plurality of angular sections of the adhesive layer. A sixth pair of the plurality of primary electrode paths may comprise the third electrode path and the fourth electrode path. Each electrode path of the sixth pair may include a respective radial part extending in a substantially radial direction towards the stomal opening in a sixth angular section of the plurality of angular sections of the adhesive layer. The radial parts of the sixth pair may be configured for allowing the provision of a sixth signal indicative of liquid on the proximal surface of the sixth angular section of the adhesive layer. The sixth signal may preferably be more indicative of liquid on the proximal surface of the sixth angular section of the adhesive layer than of liquid on the proximal surface of any other of the plurality of angular sections of the adhesive layer.

Additionally or alternatively, the number of the plurality of angular sections may equal the number of pairs of electrode paths.

Additionally or alternatively, the plurality of angular sections may together extend around the entire circumference around the stomal opening.

Additionally or alternatively, each angular section may have substantially the same angular extent. For example, if the plurality of angular sections numbers four, each angular section may extend in an angle space of 90 degrees.

Such an arrangement ensures that the entire circumference of the stomal opening is covered by angular sections and respective signals are derived therefrom to allow detection of moisture or liquid around the entire stomal opening.

Additionally or alternatively, each angular section of the plurality of angular sections may extend radially from the stomal opening. This may ensure that a leak initiated from the stomal opening enters at least one of the angular sections to allow the signal provided for that angular section to indicate elevated moisture or the presence of liquid in that angular section.

Additionally or alternatively, each angular section may extend, e.g. delimited, between the radial parts of the respective pair of electrode paths. For example, the first angular section may be delimited by the radial part of the first electrode path and the radial part of the second electrode path and so forth.

Additionally or alternatively, each of the plurality of primary electrode paths may comprise a circumferential part extending circumferentially and partially around the stomal opening, preferably the circumferential part may extend from a neck portion of the support layer to the respective angular section.

Additionally or alternatively, the angular sections may not comprise the circumferential part of any one of the plurality of electrode paths. In order words, the circumferential parts of all the electrode paths extend outside the plurality of angular sections. This may provide the advantage that a leak reaches the radial parts before the circumferential parts allowing identification of the particular angular section of the leak.

Additionally or alternatively, each radial part of the plurality of primary electrode paths may extend radially from the respective circumferential part and towards, and preferably to, the stomal opening in the respective angular section.

Additionally or alternatively, the circumferential part of each electrode path may be arranged on an outer side of the radial part of the respective electrode path.

Additionally or alternatively, each angular section may comprise the radial parts of both electrode paths of the respective pair of electrode paths and preferably the radial part of at most one electrode path of any other pair of electrode paths. For example, the first angular section may comprise the radial parts of the first and second electrode paths but not the radial parts of both of the second pair of electrode paths.

Additionally or alternatively, the ostomy appliance may have a supply state and an adapted state. The stomal opening of the ostomy appliance may have a first circumference in the supply state, and the stomal opening of the ostomy appliance may have a second circumference in the adapted state. The second circumference may be longer than the first circumference, preferably due to being cut.

Such an ostomy appliance may have the advantage of allowing the user to customise the stomal opening of the ostomy appliance to the stoma of the user thereby ensuring an improved fit of the ostomy appliance.

Additionally, the radial part(s) of at least one of the plurality of primary electrode paths may be longer in the supply state than in the adapted state, preferably due to being cut.

In addition to the above-mentioned customisation advantage, cutting the radial part(s) of the electrode paths also exposes the cut ends of the radial part(s) towards the stomal opening allowing a more radially directed measurement in contrast to a previously primarily axially directed measurement. When a leak initiates from the stomal opening, such a radially directed measurement may provide an indicative signal faster than a primarily axially directed measurement since less adhesive is present between the liquid of the leak and the cut ends of the radial part(s).

Additionally or alternatively, the ostomy appliance may comprise a plurality of secondary electrode paths preferably extending circumferentially, e.g. substantially entirely, around the stomal opening.

Accordingly, the plurality of secondary electrode paths may not be intended to provide measurements only in a particular angular section, but rather to measure around the entire stomal opening.

Additionally or alternatively, a first electrode path and a second electrode path of the plurality of secondary electrode paths may be configured for allowing the provision of a signal indicative of liquid on the proximal surface of the adhesive layer and/or indicative of a moisture level in the adhesive layer, preferably between the skin of a user and the proximal surface of the adhesive layer, preferably entirely around the stomal opening.

Accordingly, the signal of the first electrode path and second electrode path of the plurality of secondary electrode paths can provide a warning to the user that a leak is developing.

Additionally or alternatively, a third electrode path and a fourth electrode path of the plurality of secondary electrode paths are configured for allowing the provision of a signal indicative of the moisture content of the adhesive layer.

Accordingly, the signal of the third electrode path and fourth electrode path of the plurality of secondary electrode paths is indicative of the remaining lifetime of the adhesive layer of the ostomy appliance.

Additionally or alternatively, the plurality of secondary electrode paths may be arranged on a radially outer side of the plurality of primary electrode paths.

Accordingly, the signal of the first electrode path and second electrode path of the plurality of secondary electrode paths can provide a last warning to the user that a leak is imminent.

Additionally or alternatively, the support layer may preferably be moisture permeable.

Additionally or alternatively, the ostomy appliance may comprise a base plate. The base plate may include the support layer, the plurality of primary electrode paths, the adhesive layer, and preferably the plurality of secondary electrode paths.

Additionally or alternatively, the ostomy appliance may comprise a sensor patch having a distal side and a proximal side. The distal side may be adapted for attachment to an adhesive surface of a base plate. The proximal side may be adapted for attachment to a skin surface of a user. The sensor patch may include the support layer, the plurality of primary electrode paths, the adhesive layer, and preferably the plurality of secondary electrode paths.

A second aspect of the present disclosure relates to a method of manufacturing an ostomy appliance according to the first aspect of this disclosure, the method comprising the steps of:
providing a support layer with a distal surface and a proximal surface;
arranging a plurality of primary electrode paths on the proximal surface of the support layer; and
arranging an adhesive layer on the proximal surface of the support layer so as to cover the plurality of primary electrode paths.

A third aspect of this disclosure relates to a method of adapting a stomal opening of an ostomy appliance according to the first aspect of the present disclosure to the stoma of a user, the method comprising the steps of:
providing an ostomy appliance in a supply state, wherein the stomal opening of the ostomy appliance in the supply state has a first circumference,
bringing the ostomy appliance to an adapted state by changing the first circumference of the stomal opening to a second circumference of the stomal opening being longer than the first circumference, thereby bringing the ostomy appliance to an adapted state.

Additionally, the step of bringing the ostomy appliance to an adapted state may be performed by cutting the stomal opening to provide the stomal opening with the second circumference.

A person skilled in the art will appreciate that any one or more of the above aspects of this disclosure and embodiments thereof may be combined with any one or more of the other aspects of this disclosure and embodiments thereof.

### BRIEF DESCRIPTION OF THE DRAWINGS

Embodiments of this disclosure will be described in more detail in the following with regard to the accompanying figures. The figures show one way of implementing the present invention and are not to be construed as being limiting to other possible embodiments falling within the scope of the attached claim set.

FIG. 1 schematically illustrates an exploded view of an exemplary two-piece ostomy appliance.

FIG. 2 schematically illustrates an exploded view of an exemplary sensor patch of a two-piece ostomy appliance.

FIG. 3 schematically illustrates an exemplary sensor patch of a two-piece ostomy appliance.

FIG. 4 schematically illustrates an exemplary electrode path configuration.

### DETAILED DESCRIPTION OF THE INVENTION

In the following detailed description, reference is made to the accompanying drawings, which form a part hereof, and in which is shown by way of illustration specific embodiments in which the invention may be practiced. In this regard, directional terminology, such as “top,” “bottom,” “front,” “back,” “leading,” “trailing,” etc., is used with respect to the orientation of the Figure(s) being described. Because components of embodiments can be positioned in a number of different orientations, the directional terminology is used for purposes of illustration and is in no way limiting. It is to be understood that other embodiments may be utilized, and structural or logical changes may be made without departing from the scope of the present invention. The following detailed description, therefore, is not to be taken in a limiting sense, and the scope of the present invention is defined by the appended claims.

It is to be understood that the features of the various exemplary embodiments described herein may be combined with each other, unless specifically noted otherwise.

Throughout this disclosure, the words “stoma” and “ostomy” are used to denote a surgically created opening bypassing the intestines or urinary tract system of a person. The words are used interchangeably, and no differentiated meaning is intended. The same applies for any words or phrases derived from these, e.g. “stomal”, “ostomies” etc. Also, the solid and liquid wastes emanating from the stoma may be referred to as both stomal “output”, “waste(s)”, and “fluids” interchangeably. A subject having undergone ostomy surgery may be referred to as “ostomist” or “ostomate”—moreover, also as “patient” or “user”. However, in some cases “user” may also relate or refer to a health care professional (HCP), such as a surgeon or an ostomy care nurse or others.

In those cases, it will either be explicitly stated or be implicit from the context that the “user” is not the “patient” him- or herself.

In the following, whenever referring to a proximal side of a device or part of a device, the referral is to the skin-facing side, when the ostomy appliance is worn by a user. Likewise, whenever referring to the distal side of a device or part of a device, the referral is to the side facing away from the skin, when the ostomy appliance is worn by a user. In other words, the proximal side is the side closest to the user when the appliance is fitted on a user, and the distal side is the opposite side—the side furthest away from the user when the appliance is fitted on the user.

The axial direction is defined as the direction of the stoma when the appliance is worn by a user. Thus, the axial direction is generally perpendicular to the skin or abdominal surface of the user.

The radial direction is defined as transverse to the axial direction that is transversely to the direction of the stoma, i.e. “across” the distal/proximal surface of the base plate. In some sentences, the words “inner” and “outer” may be used. These qualifiers should generally be perceived with respect to the radial direction, such that a reference to an “outer” element means that the element is farther away from a centre portion of the ostomy appliance than an element referenced as “inner”. In addition, “innermost” should be interpreted as the portion of a component forming a centre of the component and/or being adjacent to the centre of the component. In analogy, “outermost” should be interpreted as a portion of a component forming an outer edge or outer contour of a component and/or being adjacent to that outer edge or outer contour.

The use of the word “substantially” as a qualifier to certain features or effects in this disclosure is intended to simply mean that any deviations are within tolerances that would normally be expected by the skilled person in the relevant field.

The use of the word “generally” as a qualifier to certain features or effects in this disclosure is intended to simply mean—for a structural feature: that a majority or major portion of such feature exhibits the characteristic in question, and—for a functional feature or an effect: that a majority of outcomes involving the characteristic provide the effect, but that exceptionally outcomes do not provide the effect.

The use of the word “essentially” as a qualifier to certain structural and functional features or effects in this disclosure is used for emphasizing what is the most important focus of something or fact about something (i.e. a feature may have or fulfil a variety of effects, but when the disclosure discusses one effect as being “essentially” provided, this is the focus and the most important effect in relation to the disclosure).

Throughout the disclosure, the use of the terms “first”, “second”, “third”, “fourth”, “primary”, “secondary”, “tertiary” etc. does not imply any particular order or importance but is included merely to identify individual elements. Furthermore, the labelling of a first element does not imply the presence of a second element and vice versa.

Disclosed is an ostomy appliance which can either be a one-piece ostomy appliance or a two-piece ostomy appliance. The ostomy appliance comprises a base plate and an ostomy pouch (also referred to as an ostomy bag). The base plate is configured for coupling to a user's stoma and/or skin surrounding the stoma, such as a peristomal skin area. The ostomy appliance may be a colostomy appliance, an ileostomy appliance or a urostomy appliance. The ostomy appliance may be a two-piece ostomy appliance, i.e. the base plate and the ostomy pouch may be releasably coupled e.g. with a mechanical and/or an adhesive coupling, e.g. to allow that a plurality of ostomy pouches can be utilized (exchanged) with one base plate. For example, the base plate may comprise a coupling ring for coupling an ostomy pouch to the base plate. Further, a two-piece ostomy appliance may facilitate correct application of the base plate to skin, e.g. to an improved user sight of the stomal region. Alternatively, the ostomy appliance may be a one-piece ostomy appliance, i.e. the base plate and the ostomy pouch may be fixedly attached to each other. The one-piece or two-piece ostomy appliance may further include a separate sensor patch for attachment to the base plate. The sensor patch may facilitate detection of moisture propagation in an adhesive material as well as detection of a heightened risk of leakage. For example, the sensor patch may allow electronic measurements of performance of the base plate and/or to facilitate detection of increasing risks of leakage and/or to facilitate detection of decreasing adherence of the base plate to the skin of the user.

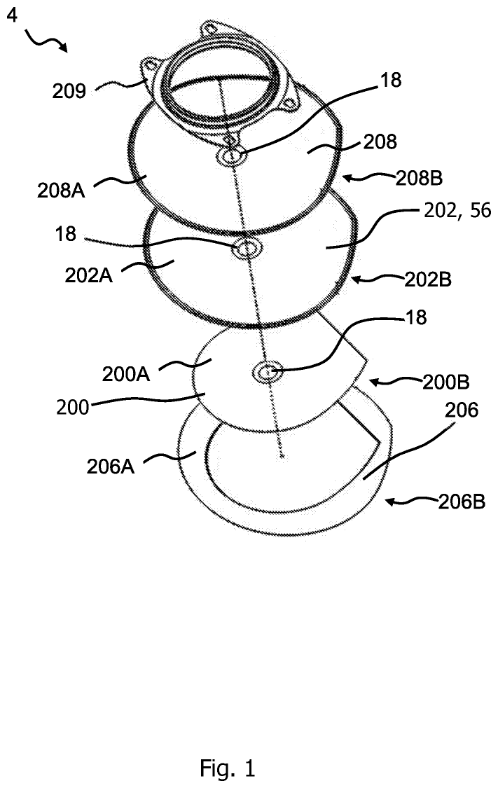

FIG. 1 schematically illustrates an exploded view of an exemplary two-piece ostomy appliance comprising a base plate 4. The base plate 4 comprises a first adhesive layer 200 having a distal surface 200A and a proximal surface 200B. During use, the proximal surface 200B of the first adhesive layer 200 adheres to the user's skin. The base plate 4 comprises a second adhesive layer 202 having a distal surface 202A and a proximal surface 202B. As illustrated, the second adhesive layer 202 spans a larger surface area than the first adhesive layer 200, such as to provide a rim of the proximal surface 202B of the second adhesive layer 202 surrounding the proximal surface 200B of the first adhesive layer 200.

The base plate 4 comprises a release liner 206, which may be peeled off by the user prior to applying the base plate 4 to the skin. The release liner 206 comprises a distal surface 206A and a proximal surface 206B. The distal surface 206A of the release liner 206 is covering the proximal surface of the first adhesive layer 200 and covering the proximal surface of the second adhesive layer 202 not covered by the first adhesive layer 200.

The base plate 4 comprises a backing layer 208. The backing layer 208 is a protective layer protecting the adhesive layers, such as the first adhesive layer 200 and/or the second adhesive layer 202 from external strains and stress during use. Furthermore, the backing layer 208 also covers the adhesive layers, such as the first adhesive layer 200 and/or the second adhesive layer 202, such that the adhesive layers 200, 202 do not adhere to clothes worn on top of the base plate 4. The backing layer 208 comprises a distal surface 208A and a proximal surface 208B. The distal surface 208A of the backing layer 208 is configured to face away from the skin of the user. The proximal surface 208B of the backing layer 208 is covering the second adhesive layer 202.

The base plate 4 forms part of a two-piece ostomy appliance, thus comprising a coupling ring 209 for coupling an ostomy pouch to the base plate 4, such as to a distal side of the base plate 4.

The base plate 4 comprises a stomal opening. The layers of the base plate 4, such as the first adhesive layer 200, the second adhesive layer 202 and the backing layer 208 as illustrated, may comprise stomal openings 18 for collectively forming the stomal opening of the base plate.

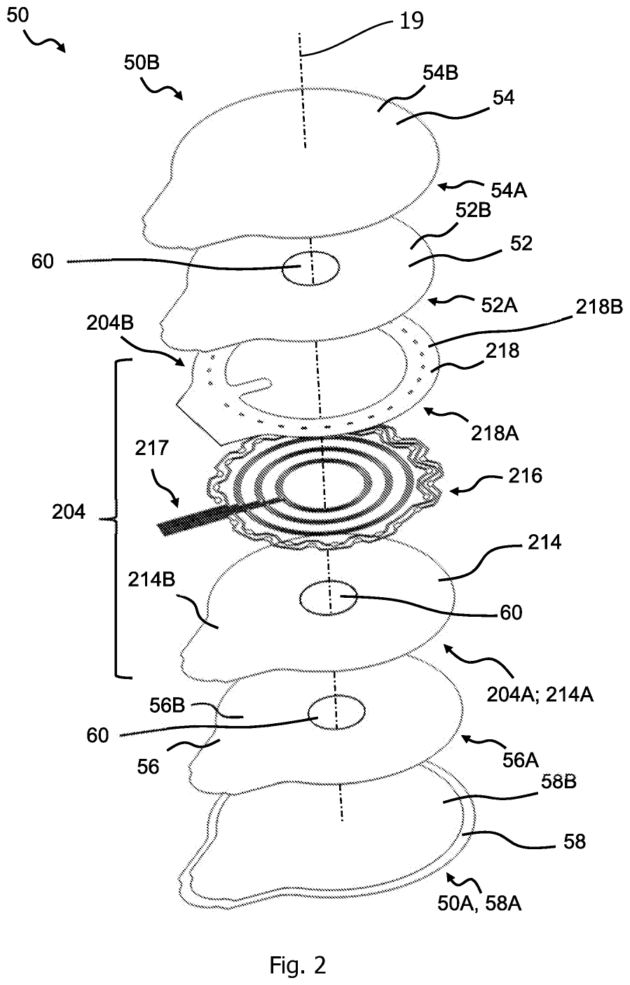

FIG. 2 schematically illustrates an exploded view of exemplary two-piece ostomy appliances comprising a sensor patch 50, such as a sensor patch 50 being adapted for attachment to a base plate, such as the base plate 4 as illustrated in FIG. 1. The sensor patch 50 is configured to be positioned between the skin of the user and the proximal side of the base plate 4. For example, the sensor patch may be adapted for attachment to the first adhesive layer 200, such as the proximal surface 200B of the first adhesive layer 200, of the base plate 4. The sensor patch 50 is configured to be attached to the base plate such that the distal side 50A of the sensor patch 50 is attached to the proximal side of the base plate, such as to the proximal surface 200B of the first adhesive layer 200 of the base plate 4.

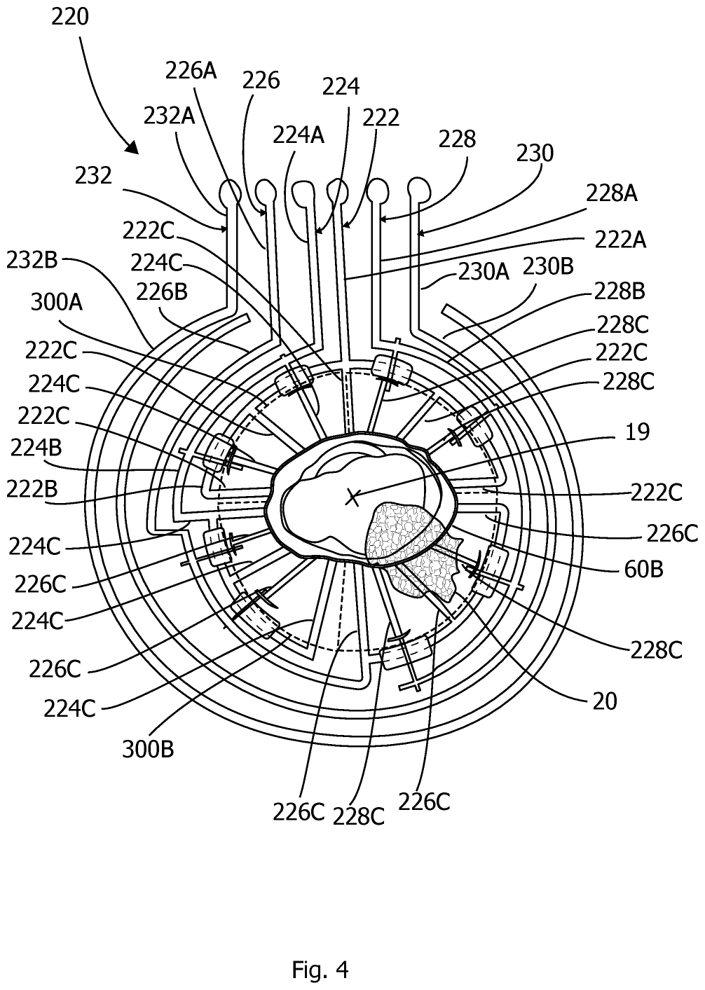

The sensor patch 50 comprises a sensor assembly 204 comprising a plurality of electrode paths 216 including for example, as shown in FIG. 4, a first electrode path 222, a second electrode path 224, a third electrode path 226, a fourth electrode path 228, a fifth electrode path 230, a sixth electrode path 232. Each electrode path 216 has respective connection parts 217 for connecting the plurality of electrode paths 216 to respective terminal elements of a monitor device. The sensor assembly 204 may form a sensor assembly layer. It is envisaged that the electrode configuration shown in FIG. 4 can replace the electrode configuration 220 of the plurality of electrode paths 216 shown in FIG. 2.

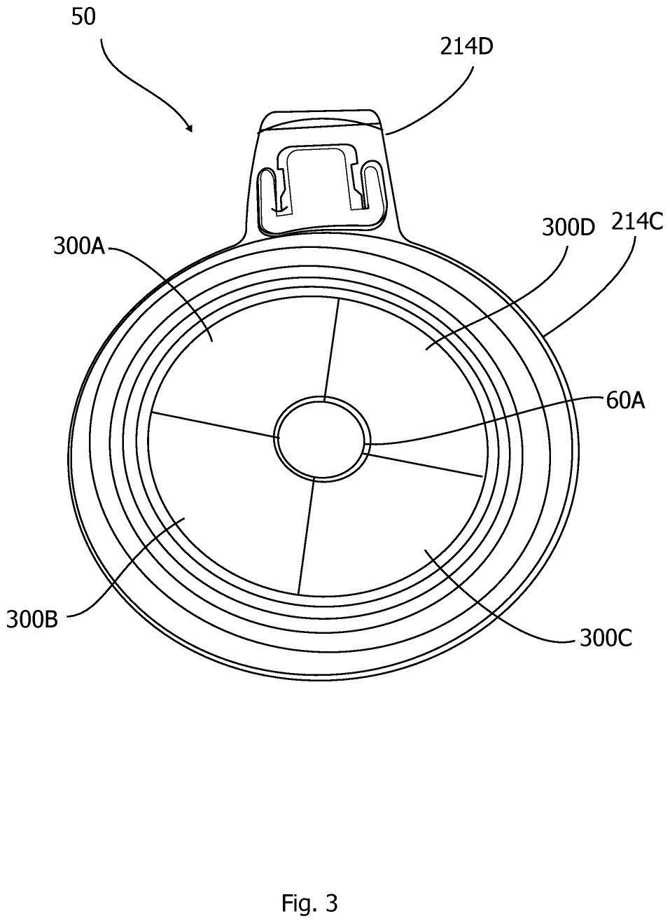

The sensor assembly 204 has a distal side 204A and a proximal side 204B. The sensor assembly 204 comprises a support layer 214 with a distal surface 214A and a proximal surface 214B. The electrode paths 216 may be provided, such as formed, on the proximal surface 214B of the support layer 214, e.g. the electrode paths 216 may be positioned on the proximal surface 214B of the support layer 214 as shown in FIG. 2. The support layer 214 comprises a ring portion 214C extending around the entire stomal opening 60 and a neck portion 214D extending radially from the ring portion 214C, see FIG. 3.

The electrode path assembly 204 may further comprise a masking element 218 having a distal surface 218A and a proximal surface 218B as shown in FIG. 2 but not in FIGS. 3-4. The masking element 218 is configured to electrically insulate at least parts of the electrode paths 216 from adjacent layers, such as a first adhesive sensor layer 52 as shown in FIG. 2. The masking element 218 covers or overlaps with parts of the electrode paths 216 when seen in the axial direction.

The sensor patch 50 comprises the first adhesive sensor layer 52 with a proximal side 52B and a distal side 52A. The first adhesive sensor layer 52 is arranged on the proximal side 204B of the sensor assembly 204 and covering the neck portion 214D of the support layer 214. The proximal side 52B of the first adhesive sensor layer 52 is configured to adhere to the user's skin. Thus, after being applied to the base plate, the combined base plate and sensor patch 50 form an adhesive proximal surface configured to be applied to the skin surface of the user.

The sensor patch comprises a first sensor release liner 54. The first sensor release liner 54 may comprise a distal surface 54A and a proximal surface 54B. The first sensor release liner 54 may be arranged to protect the first adhesive sensor layer 52. The distal surface 54A of the first sensor release liner 54 is facing the proximal surface 52B of the first adhesive sensor layer 52. The first sensor release liner 54 is configured to be peeled off by the user prior to application of the base plate with the attached sensor patch to the skin. The first adhesive sensor layer 52 may be laid out on the distal side 54A of the first sensor release liner 54.

The sensor patch 50 may comprise a second adhesive sensor layer 56, with a proximal side 56B and a distal side 56A. The second adhesive sensor layer 56 is arranged on the distal side 204A of the sensor assembly 204 but does not cover the neck portion 214D of the support layer 214. The proximal side 56B of the second adhesive sensor layer 52 is configured to adhere to the base plate, such as the proximal surface of the first adhesive layer of the base plate.

The sensor patch 50 comprises a stomal opening 60. The layers of the sensor patch 50, such as the first adhesive sensor layer 52, the support layer 214 and the second adhesive sensor layer 56, as illustrated, may comprise stomal openings 60 for collectively forming the stomal opening of the sensor patch 50. The stomal opening 60 of the sensor patch is configured to be aligned with the stomal opening 18 of the base plate, such as to collectively form the stomal opening of the combined base plate and sensor patch 50.

As shown in FIGS. 2 and 3, the sensor patch 50 comprises a stomal opening 60 with a centre point 19 (in FIG. 2 illustrated as by a centre line). In some exemplary sensor patches, the stomal opening 60 may be made by the user, i.e. the sensor patch may be manufactured and/or sold without the stomal opening 60, but with a region adapted to form the stomal opening 60 with the centre point 19.

The exemplary sensor patch comprises a second sensor release liner 58. The second sensor release liner 58 may comprise a distal surface 58A and a proximal surface 58B. The second sensor release liner 58 may be arranged to protect the second adhesive sensor layer 56. The proximal surface 58B of the second sensor release liner 58 is facing the distal surface 56A of the second adhesive sensor layer 56. The second sensor release liner 58 is configured to be peeled off by the user prior to application of the sensor patch to the base plate. The second adhesive sensor layer 56 may be laid out on the proximal side 58B of the second sensor release liner 58.

FIG. 3 schematically illustrates an exemplary sensor patch 50 for an ostomy appliance similar to the sensor patch shown in FIG. 2. The sensor patch 50 comprises four angular sections 300A, 300B, 300C, 300D. Four leakage sensors are configured to provide signal indicative of a leak (e.g. increased moisture levels or liquid on between the skin surface of the user and the sensor patch 50) in each respective angular section. Each angular section extends radially from the stomal opening 60A and thus ensures that a leak initiated from the stomal opening 60A enters at least one of the angular sections and allows the signal provided for that angular section to indicate elevated moisture or the presence of liquid in that angular section. Each angular section has substantially the same angular extent. In the present case, each angular section extends in an angle space of 90 degrees.

Further, the sensor patch 50 has a supply state and an adapted state. FIG. 3 shows the sensor patch 50 in the supply state. In the supply state, the stomal opening 60A of the sensor patch 50 has a first circumference. FIG. 4 schematically illustrates an exemplary electrode path configuration 220 of electrode paths 222, 224, 226, 228, 230, 232 of the sensor patch shown in FIG. 3. FIG. 4 also illustrates the circumference of the stomal opening 60B in the adapted state of the sensor patch of FIG. 3. As seen, the second circumference is longer than the first circumference due to being cut to adapt the stomal opening 60B to the stoma of the user.

Furthermore, it is envisioned that a second alternative ostomy appliance according to the disclosure may be provided with the sensor patch 50 described in connection with FIG. 3 incorporated in a base plate 4. In such a second alternative ostomy appliance, the first adhesive layer 200 or the second adhesive layer 202 of the base plate as described in connection with FIG. 1 may further perform the functions of the second adhesive sensor layer 56, and the support layer 214 may be arranged between the first adhesive layer 200 and the second adhesive layer 202. The second adhesive layer 202 thus covers the plurality of electrodes 222, 224, 226, 228, 230, 232 being arranged on the distal side 214B of the support layer, and the second adhesive layer 202 comprises the inner ring portion 56C and the outer ring portion 56D.

FIG. 4 schematically illustrates an exemplary electrode path configuration 220 of electrode paths 216 of the sensor patch shown in FIG. 3. The plurality of electrode paths 216 comprises a first electrode path 222, a second electrode path 224, a third electrode path 226, a fourth electrode path 228, a fifth electrode path 230, and a sixth electrode path 232.

The first electrode path 222 comprises a first connection part 222A, a first circumferential part 222B, and first radial parts 222C. The second electrode path 224 comprises a second connection part 224A, a second circumferential part 224B, and second radial parts 224C. The third electrode path 226 comprises a third connection part 226A, a third circumferential part 226B, and third radial parts 226C. The fourth electrode path 228 comprises a fourth connection part 228A, a fourth circumferential part 228B, and fourth radial parts 228C. The fifth electrode path 230 comprises a fifth connection part 230A and a fifth circumferential part 230B. The sixth electrode path 232 comprises a sixth connection part 232A and a sixth circumferential part 232B.

The connection parts 222A, 224A, 226A, 228A, 230A, 232A extend from the respective circumferential parts 222B, 224B, 226B, 228B, 230B, 232B to the neck portion 214D of the support layer 214 as seen in FIG. 3 and thus allow forming an electrical connection between different pairs of electrode paths. The circumferential parts 222B, 224B, 226B, 228B, 230B, 232B extend circumferentially around the stomal opening 60B. The radial parts 222C, 224C, 226C, 228C extend in respective radial directions from the respective circumferential parts to the circumference of the stomal opening 60B, and in particular in a direction towards the centre point 19 of the stomal opening 60B.

The first radial parts 222C of the first electrode path 222 and the second radial parts 224C of the second electrode path 224 are configured for allowing the provision of a first signal indicative of liquid on the proximal surface and/or of a moisture level of the first angular section 300A. Accordingly, the first angular section 300A is delimited by the angular extent of the first and second radial parts 222C, 224C.

The second radial parts 224C of the second electrode path 224 and the third radial parts 226C of the third electrode path 226 are configured for allowing the provision of a second signal indicative of liquid on the proximal surface and of a moisture level of the second angular section 300B. Accordingly, the second angular section 300B is delimited by the angular extent of the second and third radial parts 224C, 226C.

The third radial parts 226C of the third electrode path 226 and the fourth radial parts 228C of the fourth electrode path 228 are configured for allowing the provision of a third signal indicative of liquid on the proximal surface and of a moisture level of the third angular section 300C. Accordingly, the third angular section 300C is delimited by the angular extent of the third and fourth radial parts 226C, 228C.

The first radial parts 222C of the first electrode path 222 and the fourth radial parts 228C of the fourth electrode path 228 are configured for allowing the provision of a fourth signal indicative of liquid on the proximal surface and of a moisture level of the fourth angular section 300D. Accordingly, the fourth angular section 300D is delimited by the angular extent of the first and fourth radial parts 222C, 228C.

Accordingly, the radial parts 222C of the first electrode path 222 extend within the first and fourth angular sections 300A, 300D but not in the remaining angular sections. The radial parts 224C of the second electrode path 224 extend within the first and second angular sections 300A, 300B but not in the remaining angular sections. The radial parts 226C of the third electrode path 226 extend in the second and third angular sections 300B, 300C but not in the remaining angular sections. Lastly, the radial parts 228C of the fourth electrode path 228 extend in the third and fourth angular sections 300C, 300D but not in the remaining angular sections.

The first, second, third and fourth signals are provided by measuring an electrical resistance between the respective electrode paths. An electrically insulating material insulates different electrode paths at each crossing of a radial part and a circumferential part.

The circumferential parts 230B, 232B of the fifth and sixth electrode paths 230, 232 extend circumferentially substantially entirely around the stomal opening 60B on a radially outer side of the circumferential parts 222B, 224B, 226B, 228B of the first, second, third and fourth electrode paths 222, 224, 226, 228. The fifth and sixth electrode paths 230, 232 do not comprise radial parts extending from the circumferential parts towards the stomal opening 60B. Accordingly, the fifth and sixth electrode paths 230, 232 are not intended to provide measurements only in a particular angular section, but rather measure around the entire stomal opening e.g. for providing a signal indicative leakage and/or moisture content in adhesive.

| | |
|---|---|
| 4 | base plate |
| 18 | stomal opening |
| 19 | centre point |
| 20 | leak |
| 50 | sensor patch |
| 50A | distal side |
| 50B | proximal side |
| 52 | first adhesive sensor layer |
| 52A | distal side |
| 52B | proximal side |
| 54 | first sensor release liner |
| 54A | distal surface |
| 54B | proximal surface |
| 56 | second adhesive sensor layer |
| 56A | distal side |
| 56B | proximal side |
| 56C | inner ring portion |
| 56D | outer ring portion |
| 58 | second sensor release liner |
| 58A | distal surface |
| 58B | proximal surface |
| 60 | stomal opening |
| 60A | supply state of stomal opening |
| 60B | adapted state of stomal opening |
| 200 | first adhesive layer |
| 200A | distal surface |
| 200B | proximal surface |
| 202 | second adhesive layer |
| 202A | distal surface |
| 202B | proximal surface |
| 204 | sensor assembly |
| 204A | distal side |
| 204B | proximal side |
| 206 | release liner |
| 206A | distal surface |
| 206B | proximal surface |
| 208 | backing layer |
| 208A | distal surface |
| 208B | proximal surface |
| 209 | coupling ring |
| 214 | support layer |
| 214A | distal surface |
| 214B | proximal surface |
| 214C | ring portion |
| 214D | neck portion |
| 216 | electrode path |
| 217 | connection part |
| 218 | masking element |
| 218A | distal surface |
| 218B | proximal surface |
| 220 | electrode path configuration |
| 222 | first electrode part |
| 222A | first connection part |
| 222B | first circumferential part |
| 222C | first radial part |
| 224 | second electrode part |
| 224A | second connection part |
| 224B | second circumferential part |
| 224C | second radial part |
| 226 | third electrode part |
| 226A | third connection part |
| 226B | third circumferential part |
| 226C | third radial part |
| 228 | fourth electrode part |
| 228A | fourth connection part |
| 228B | fourth circumferential part |
| 228C | fourth radial part |
| 230 | fifth electrode part |
| 230A | fifth connection part |
| 230B | fifth circumferential part |
| 232 | sixth electrode path |
| 232A | sixth connection part |
| 232B | sixth circumferential part |
| 300A | first angular section |
| 300B | second angular section |
| 300C | third angular section |
| 300D | fourth angular section |

1. An ostomy appliance for attachment to the skin surface of a user, wherein the ostomy appliance is adapted to form a stomal opening with a centre point, the stomal opening being configured to allow passage of output through the stomal opening and into an ostomy pouch attached to the ostomy appliance, the ostomy appliance comprising:
a support layer with a distal surface and a proximal surface;
an adhesive layer including a distal surface and a proximal surface, the distal surface being arranged on the proximal surface of the support layer; and
a plurality of primary electrode paths for forming one or more sensors, the plurality of primary electrode paths comprising at least a first electrode path, a second electrode path, and a third electrode path, wherein the plurality of primary electrode paths is arranged on the proximal surface of the support layer and covered by the adhesive layer;
wherein a first pair of the plurality of primary electrode paths comprises the first electrode path and the second electrode path, wherein each electrode path of the first pair includes a respective radial part extending in a substantially radial direction towards the stomal opening in a first angular section of a plurality of angular sections of the adhesive layer, the radial parts of the first pair being configured for allowing the provision of a first signal indicative of liquid on the proximal surface of the first angular section of the plurality of angular sections of the adhesive layer and/or indicative of a moisture level in the first angular section of the plurality of angular sections of the adhesive layer,
wherein a second pair of the plurality of primary electrode paths comprises at least the third electrode path, each electrode path of the second pair includes a respective radial part extending in a substantially radial direction towards the stomal opening in a second angular section of the plurality of angular sections of the adhesive layer, the radial parts of the second pair being configured for allowing the provision of a second signal indicative of liquid on the proximal surface of the second angular section of the plurality of angular sections of the adhesive layer and/or indicative of a moisture level in the second angular section of the plurality of angular sections of the adhesive layer.

2. The ostomy appliance according to claim 1, wherein the first signal is more indicative of liquid on the proximal surface of a first angular section of the plurality of angular sections of the adhesive layer than of the second angular section of the adhesive layer, and wherein the second signal is more indicative of liquid on the proximal surface of the second angular section of the plurality of angular sections of the adhesive layer than of the first angular section of the adhesive layer.

3. The ostomy appliance according claim 1, wherein the radial parts of the first electrode path and the second electrode path extend in the first angular section, and the radial part(s) of at least one of the first electrode path and the second electrode path do not extend in the second angular section, and
wherein the radial parts of both of the electrode paths of the second pair of the plurality of primary electrode paths extend in the second angular section, and the radial part(s) of at least one electrode path of the second pair of the plurality of primary electrode paths do not extend in the first angular section.

4. The ostomy appliance according to claim 1, wherein the second pair of the plurality of primary electrode paths comprises the first electrode path and the third electrode path, and
a third pair of the plurality of primary electrode paths comprises the second electrode path and the third electrode path,
wherein each electrode path of the third pair includes a respective radial part extending in a substantially radial direction towards the stomal opening in a third angular section of the plurality of angular sections of the adhesive layer, the radial parts of the third pair is configured for allowing the provision of liquid on the proximal surface of the third angular section of the plurality of angular sections of the adhesive layer.

5. The ostomy appliance according to claim 4, wherein the plurality of primary electrode paths includes a fourth electrode path,
wherein a fourth pair of the plurality of primary electrode paths comprises the first electrode path and the fourth electrode path and each electrode path of the fourth pair includes a respective radial part extending in a substantially radial direction towards the stomal opening in a fourth angular section of the plurality of angular sections of the adhesive layer, the radial parts of the fourth pair being configured for allowing the provision of a fourth signal indicative of liquid on the proximal surface of the fourth angular section of the adhesive layer,
wherein a fifth pair of the plurality of primary electrode paths comprises the second electrode path and the fourth electrode path and each electrode path of the fifth pair includes a respective radial part extending in a substantially radial direction towards the stomal opening in a fifth angular section of the plurality of angular sections of the adhesive layer, the radial parts of the fifth pair being configured for allowing the provision of a fifth signal indicative of liquid on the proximal surface of the fifth angular section of the adhesive layer,
wherein a sixth pair of the plurality of primary electrode paths comprises the third electrode path and the fourth electrode path and each electrode path of the sixth pair includes a respective radial part extending in a substantially radial direction towards the stomal opening in a sixth angular section of the plurality of angular sections of the adhesive layer, the radial parts of the sixth pair being configured for allowing the provision of a sixth signal indicative of liquid on the proximal surface of the sixth angular section of the adhesive layer.

6. The ostomy appliance according to claim 1, wherein the number of the plurality of angular sections equals the number of pairs of electrode paths, and the plurality of angular sections together extends around the entire circumference around the stomal opening.

7. The ostomy appliance according to claim 1, wherein each of the plurality of primary electrode paths comprises a circumferential part extending circumferentially and partially around the stomal opening.

8. The ostomy appliance according to claim 7, wherein each radial part of the plurality of primary electrode paths extends radially from the respective circumferential part and towards the stomal opening in the respective angular section.

9. The ostomy appliance according to claim 8, wherein ostomy appliance has a supply state and an adapted state, wherein the stomal opening of the ostomy appliance in the supply state has a first circumference, and the stomal opening of the ostomy appliance in the adapted state has a second circumference being longer than the first circumference.

10. The ostomy appliance according to claim 9, wherein the radial part(s) of at least one of the plurality of primary electrode paths is longer in the supply state than in the adapted state.

11. The ostomy appliance according to claim 1, further comprising a plurality of secondary electrode paths.

12. The ostomy appliance according to claim 11, wherein a first electrode path and a second electrode path of the plurality of secondary electrode paths are configured for allowing the provision of a signal indicative of liquid on the proximal surface of the adhesive layer.

13. The ostomy appliance according to claim 11, wherein a third electrode path and a fourth electrode paths of the plurality of secondary electrode paths are configured for allowing the provision of a signal indicative of the moisture content of the adhesive layer.

14. The ostomy appliance according to claim 11, any one of claims 11-13, wherein the plurality of secondary electrode paths is arranged on a radially outer side of the plurality of primary electrode paths.

15. The ostomy appliance according to claim 1, wherein the ostomy appliance comprises a base plate, the base plate including the support layer, the plurality of primary electrode paths, the adhesive layer, and preferably the plurality of secondary electrode paths.

16. The ostomy appliance according to claim 1, wherein the ostomy appliance comprises a sensor patch having a distal side and a proximal side, the distal side being adapted for attachment to an adhesive surface of a base plate, and the proximal side being adapted for attachment to a skin surface of a user, the sensor patch including the support layer, the plurality of primary electrode paths, the adhesive layer, and preferably the plurality of secondary electrode paths.

17. A method of manufacturing an ostomy appliance, wherein the ostomy appliance is adapted to form a stomal opening with a centre point, the stomal opening being configured to allow passage of output through the stomal opening and into an ostomy pouch attached to the ostomy appliance, the method comprising the steps of:
providing a support layer with a distal surface and a proximal surface;
arranging a plurality of primary electrode paths on the proximal surface of the support layer, the plurality of primary electrode paths comprising at least a first electrode path, a second electrode path, and a third electrode path; and
arranging an adhesive layer on the proximal surface of the support layer so as to cover the plurality of primary electrode paths,
wherein a first pair of the plurality of primary electrode paths comprises the first electrode path and the second electrode path, wherein each electrode path of the first pair includes a respective radial part extending in a substantially radial direction towards the stomal opening in a first angular section of a plurality of angular sections of the adhesive layer, the radial parts of the first pair being configured for allowing the provision of a first signal indicative of liquid on the proximal surface of the first angular section of the plurality of angular sections of the adhesive layer and/or indicative of a moisture level in the first angular section of the plurality of angular sections of the adhesive layer, and
wherein a second pair of the plurality of primary electrode paths comprises at least the third electrode path, each electrode path of the second pair includes a respective radial part extending in a substantially radial direction towards the stomal opening in a second angular section of the plurality of angular sections of the adhesive layer, the radial parts of the second pair being configured for allowing the provision of a second signal indicative of liquid on the proximal surface of the second angular section of the plurality of angular sections of the adhesive layer and/or indicative of a moisture level in the second angular section of the plurality of angular sections of the adhesive layer.

18. A method of adapting a stomal opening of an ostomy appliance, wherein the ostomy appliance is adapted to form a stomal opening with a centre point, the stomal opening being configured to allow passage of output through the stomal opening and into an ostomy pouch attached to the ostomy appliance, the method comprising:
providing an ostomy appliance in a supply state, wherein the stomal opening of the ostomy appliance in the supply state has a first circumference,
bringing the ostomy appliance to an adapted state by changing the first circumference of the stomal opening to a second circumference of the stomal opening being longer than the first circumference, thereby bringing the ostomy appliance to an adapted state.

19. The method according to claim 18, wherein the step of bringing the ostomy appliance to an adapted state is performed by cutting the stomal opening to provide the stomal opening with the second circumference.
